(19) United States
(12) Patent Application Publication
(10) Pub. No.: US 2020/0330470 A1
(43) Pub. Date: Oct. 22, 2020

(54) FILM FORMULATION COMPRISING VARDENAFIL, METHOD FOR ITS PREPARATION, AND USE THEREOF

(71) Applicant: KLARIA PHARMA HOLDING AB, UPPSALA (SE)

(72) Inventors: Fredrik HÜBINETTE, UPPSALA (SE); Erik MASCHER, SIGTUNA (SE)

(21) Appl. No.: 16/772,788

(22) PCT Filed: Dec. 20, 2018
(86) PCT No.: PCT/EP2018/086406

§ 371 Date: Jun. 14, 2020

(30) Foreign Application Priority Data
Dec. 20, 2017 (EP) 17208875.9

(51) Int. Cl. A61K 31/53; A61K 9/70; A61K 47/26; A61K 47/20; A61K 47/22

(52) U.S. Cl. CPC A61K 31/53; A61K 9/7007; A61K 47/22; A61K 47/20; A61K 47/26

(57) ABSTRACT

A method of preparing a mucoadhesive film containing vardenafil or a pharmaceutically acceptable salt of vardenafil, by admixing, in an aqueous liquid carrier, vardenafil or a pharmaceutically acceptable salt of vardenafil, a C3-C12 polyol, and a pH regulating agent, to obtain a liquid composition having a pH of at least 4.2; admixing the liquid composition with analginate salt of monovalent cation or a mixture of such salts, distributing the obtained composition onto a solid surface; and permitting the composition to dry. A mucoadhesive film containing vardenafil or a pharmaceutically acceptable salt of vardenafil, its use for treating sexual dysfunction.

### FIELD OF THE INVENTION

The present invention relates to a pharmaceutical film formulation containing the PDE5 inhibitor vardenafil (4-[2-Ethoxy-5-(4-ethylpiperazin-1-yl)sulfonyl-phenyl]-9-methyl-7-propyl-3,5,6,8-tetrazabicyclo[4.3.0]nona-3,7,9-trien-2-one) and to a method for preparing such a formulation.

### BACKGROUND OF THE INVENTION

PPDE5 inhibitors are used to block the degradative action of cGMP-specific PDE5 on cyclic GMP in the smooth muscle cells, such as cells lining the blood vessels supplying the corpus cavernosum of the penis and cells in the arterial wall smooth muscle within the lungs. Due to this action, PDE5 inhibitors are being used for the treatment of erectile dysfunction (ED), and are also explored for the treatment of other diseases smooth muscle cells, e.g. diseases involving pulmonary hypertension. Sildenafil, vardenafil and tadalafil are well-known PDE5 inhibitors sold in medicines under trade names such as Viagra, Cialis, Levitra, and Staxyn, just to mention a few.

In the treatment of ED, vardenafil is provided for oral administration in the form of a film coated tablet or as an orally disintegrating tablet. The recommended dose of vardenafil typically is 10 mg per day when administered in a film coated tablet and somewhat less when administered as an orally disintegrating tablet. If there is no response, the dose may be increased to 20 mg but if there are side effects, it may have to be reduced to 5 mg.

For both types of tablets, the medication is prescribed to be taken about 60 minutes before intercourse, which in some situations may be awkward.

Vardenafil has been associated with a number of side effects or adverse reactions, of varying degree of frequency and seriousness. The most commonly reported adverse reaction is headache. Other very common or adverse reactions are cardiovascular effects, such as flushing, palpitation, tachycardia, angina pectoris, myocardial infarction, ventricular tachyarrhythmias, and hypotension; CNS reactions such as headache, dizziness, paresthesia and dysesthesia, somnolence, sleep disorder, syncope, amnesia, and seizure; dermatologic reactions such as erythema, rash, angioedema, and allergic edema; gastrointestinal reactions, such as dyspepsia, nausea, gastrointestinal and abdominal pain, dry mouth, diarrhea, gastroesophageal reflux disease, gastritis, and vomiting; hepatic reactions, such as increase in transaminases; musculoskeletal reactions, such as back pain, increase in creatine phosphokinase (CPK), increased muscle tone and cramping, and myalgia; ocular reactions, such as visual disturbance, ocular hyperemia, visual color distortions, eye pain and eye discomfort, photophobia, increase in intraocular pressure, and conjunctivitis; as well as e.g. flu syndrome, tinnitus, vertigo, chest pain, and feeling unwell. A reduced dosage may be preferable to avoid unwanted side effects, but may lead to inadequate therapeutic effect

In international application WO 2007/073346, incorporated herein by reference, a mucoadhesive film formulation is provided, in the form of an alginate based film, wherein the film-forming agent is an alginate salt of monovalent cation or a mixture of alginate salts of monovalent cation, said film-forming agent being such that a 10% aqueous solution thereof at a temperature of 20° C. has a viscosity of 100-1000 mPas, as measured at a shear rate of 20 rpm by use of a Brookfield viscometer with a spindle No. 2.

WO 2007/073346 also mentions that alginate film containing at least one active ingredient may be prepared e.g. by dissolving the active ingredient(s) in a suitable solvent; optionally adjusting the pH of the solution of active ingredient(s) to around neutral or alkaline pH; optionally adding plasticizer and microcrystalline cellulose, as well as any other suitable physiologically and/or pharmaceutically acceptable additive; adding the film forming agent; and processing the solution so as to obtain a film.

Further, WO 2007/073346 mentions that the active ingredient(s) and the alginate(s) may be simply dissolved together in a suitable solvent or mixture of solvents whereafter the solution is processed to a film and permitted to dry, or the active ingredient may be added to the alginate solution so as to give an emulsion or suspension of active ingredient in the alginate solution.

Vardenafil, its use in therapy and processes for its preparation are described in the literature, e.g. in WO 1999024433, WO 2002050076, WO2004006894, WO 2005110420, WO2008151811, WO 2010130393, WO 2013075680, and US 2007197535 all of which are incorporated herein by reference.

### SUMMARY OF THE INVENTION

In one aspect, a mucoadhesive film is provided, comprising

(i) vardenafil or a pharmaceutically acceptable salt of vardenafil,

(ii) at least one C3-C12 polyol, and,

as a film forming agent, an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

In some preferred embodiments, the mucoadhesive film also comprises (ii) at least one of dimethyl sulfoxide and N-methyl-2-pyrrolidone.

A further aspect relates to a method of preparing a mucoadhesive film containing vardenafil or a pharmaceutically acceptable salt thereof, by

admixing, in an aqueous liquid carrier, (i) vardenafil or a pharmaceutically acceptable salt of vardenafil,

(ii) at least one C3-C12 polyol; and a pH regulating agent, to obtain a liquid composition having a pH of at least 4.2;

admixing the obtained liquid composition having a pH of at least 4.2 with an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol, and said alginate salt of monovalent cation or mixture of alginate salts of monovalent cation being such that a 10% aqueous solution thereof at a temperature of 20° C. has a viscosity of 100-1000 mPas, as measured at a shear rate of 20 rpm by use of a Brookfield viscometer with a spindle No. 2; to obtain a film-forming liquid composition;

distributing the obtained film-forming liquid composition onto a solid surface; and permitting the film-forming liquid composition to dry on said surface.

Still further aspects relate to the mucoadhesive film as provided herein for use in therapy, e.g. for use in the treatment of sexual dysfunction, preferably male sexual dysfunction; to the use of a film as provided herein in the manufacturing of a medicament for the treatment of sexual dysfunction, preferably male sexual dysfunction.

Still further aspects relate to a method for the treatment of sexual dysfunction, preferably male sexual dysfunction, such as male erectile disorder, by administering to a mammal in need of such treatment, e.g. an adult male human, a mucoadhesive film as provided herein.

Still further aspects relate to the use of a film as described herein for the manufacture of a medicament for the treatment of sexual dysfunction, preferably male sexual dysfunction, such as male erectile disorder.

### BRIEF DESCRIPTION OF THE DRAWINGS

FIG. 1 is a graph showing the % by weight of vardenafil released over time (in minutes) from film formulations of the invention containing various amounts of DMSO or NMP. A=Example 15, B=Example 14, C=Example 16, and D=Example 13.

FIG. 2 is a graph showing the % by weight of vardenafil released over time (in minutes) from film formulations of the invention containing various amounts of DMSO. E=Example 21, F=Example 18, G=Example 17, H=Example 19, and I=Example 20

FIG. 3 is a graph showing the % by weight of vardenafil released over time (in minutes) from two film formulations of the invention, viz. Examples 11 and 12.

FIG. 4 is a graph showing the variation of concentration of vardenafil (in ng/ml) in plasma of beagle dog over a 8-hour time period after administration of film formulations of the invention containing varying amounts of DMSO and/or NMP.

### DETAILED DESCRIPTION OF THE INVENTION

### Vardenafil

The mucoadhesive film of the present invention contains vardenafil or a pharmaceutically acceptable salt of vardenafil as active ingredient. Vardenafil (free base) has the structural formula

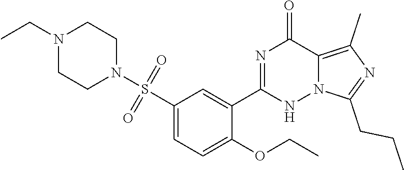 [SMILES: CCCc1nc(C)c2c(=O)nc(-c3cc(S(=O)(=O)N4CCN(CC)CC4)ccc3OCC)[nH]n12]

The compound may have the form of a free base or of a pharmaceutically acceptable salt, such an acid addition salt and any such form of vardenafil is contemplated as useful herein. However, vardenafil is generally used in the form of its hydrochloride, in particular hydrochloride trihydrate, and in some embodiments, therefore the pharmaceutically acceptable salt of vardenafil is a hydrochloride or a hydrate thereof. It should be realized, however, that the invention is not specifically limited to such salt and that any pharmaceutically acceptable salt may be used, e.g. a salt with a pharmaceutically acceptable mineral acid or organic acid.

In the following description the reference to “vardenafil” should also be construed as a reference to a pharmaceutically acceptable salt thereof, unless otherwise indicated or apparent from the context. Any reference to an amount (e.g. in grams or in % by weight) of vardenafil or a pharmaceutically acceptable salt thereof should be construed as referring to the weight free base.

### The Polyol

The mucoadhesive film of the present invention contains at least one C3-C12 polyol (also referred to as a sugar alcohol) e.g. at least one C3-C7 polyol, at least one C3-C6 polyol, or at least one C5-C6 polyol. In some embodiments, the one or more polyols are selected from C3-C7 polyols, e.g. from C3-C6 polyols, or from C3-05 polyols, or from C5-C6 polyols.

In some embodiments, the polyol has the general formula HOCH₂(CHOH)ₙCH₂OH, wherein n is an integer of from 1 to 10, or from 1 to 5, or from 1 to 4, or from 1 to 3.

In some embodiments, the polyol is selected from glycerol, erythritol, threitol, arabitol, xylitol, ribitol, mannitol, sorbitol, galactitol, fucitol, iditol, inositol, volemitol, and isomalt; e.g. from glycerol, erythritol, threitol, arabitol, xylitol, ribitol, mannitol, sorbitol, galactitol, fucitol, iditol, and inositol. In some particular embodiments, the polyol is selected from glycerol, xylitol, and sorbitol, e.g. the polyalcohol is selected from xylitol and sorbitol, or from xylitol and glycerol, or from glycerol and sorbitol.

In some embodiments, the polyol used according to the invention is xylitol and optionally one or more further polyols as defined herein above, e.g. the film contains xylitol and optionally at least one further polyol selected from glycerol and sorbitol. In some particular embodiments, the film contains the three polyols glycerol, xylitol, and sorbitol, in some embodiments, the film contains only two of these three polyols, in some embodiments the film contains only one polyol, e.g. one of the polyols defined herein above, e.g. xylitol.

The present inventor has found that the taste of vardenafil (or its salt), which may be experienced as more or less disagreeable, is effectively masked in the presence of xylitol. Therefore, in some preferred embodiments a film formulation as defined herein is provided, containing vardenafil or a pharmaceutically acceptable salt thereof, and xylitol, said film formulation having a substantially reduced taste of vardenafil, e.g. reduced to a level that is generally not perceivable to a human patient.

### The pH Regulating Agent

The pH regulating agent generally is a pharmaceutically acceptable alkaline reacting compound, or mixture of such compounds, e.g. a strong base, e.g. an alkaline reacting hydroxide of a monovalent metal cation, such as LiOH, NaOH or KOH, in particular NaOH. For example, the pH regulating agent may be added to the liquid composition in the form of a 0.01 to 5M aqueous solution, e.g. a 0.1 M to 4 M aqueous solution. For example, a 1-5 M, or 1-4 M, or 2-4 M aqueous solution of LiOH, NaOH or KOH, in particular NaOH, may be added to the aqueous solution until the required pH is reached.

### Further Ingredients

In some particular embodiments, the film contains (iii) at least one of dimethylsulfoxide (DMSO) and N-methyl-2-pyrrolidone (NMP).

In some embodiments, the film contains DMSO as an additional ingredient (iii). In some embodiments, the film contains NMP as an additional ingredient (iii). In some embodiments, the film contains a mixture of DMSO and NMP as an additional ingredient (iii).

For example, in some embodiments, the obtained film contains from about 1 to about 10% by weight of ingredient (iii), e.g. from 1.5% or from 2%, or from 3%, up to at least 9%, at least 8%, at least 7%, at least 6%, at least 5%, or at least 4%, by weight of the film (i.e. the dry film).

In some embodiments, the film contains from about 1 to about 8% by weight of ingredient (iii), e.g. from about 2 to about 6% by weight of ingredient (iii), or from about 3% by weight to about 5% by weight of ingredient (iii).

In some preferred embodiments, the film contains from about 3 to about 8% by weight of ingredient (iii), e.g. from about 3 to about 6% by weight of ingredient (iii), or from about 3% by weight to about 5% by weight of ingredient (iii).

Preferably, Ingredient (iii) is DMSO.

In some other embodiments, the film contains from about 1 to about 10% by weight of a mixture of DMSO and NMP, e.g. from 1.5% or from 2%, or from 3%, up to at least 10%, or at least 9%, or at least 8%, by weight of the film. For example, a mixture of DMSO and NMP may contain the two compounds in a weight ratio to each other of from 1:10 to 10:1, or from 1:5 to 5:1, or from 1:2 to 2:1, e.g. about 1:1.

Without wishing to be bound to any theory, it is considered that DMSO and NMP act as cosolvents for vardenafil in the film and possibly also as a mucosal permeation enhancer for the vardenafil.

In some embodiments, the film contains one or more further ingredients, e.g. one or more further therapeutically active ingredients, or one or more excipients. For example, the film may contain a colorant, such a titanium oxide, a flavoring agent, such as menthol, orange flavor, lemon flavor etc., a filler (bulking agent), such as microcrystalline cellulose, a chelating agent, such as EDTA (ethylenediaminetetraacetic acid) or a pharmaceutically acceptable salt thereof, e.g. EDTA disodium salt dihydrate, a surfactant, etc. Thus, in some embodiments, such further ingredients are present in an amount of 0-20%, e.g. 5-10% by weight of the total pharmaceutical composition. In some embodiments, the film contains no further ingredients, i.e. the film contains only the active ingredient (vardenafil or a pharmaceutically acceptable salt thereof), the polyol(s), e.g. 1-3 polyols as defined herein above, and the alginate as a film forming agent. In some embodiments, the film contains only a cosolvent as defined herein above as further ingredient.

In some embodiments, when the film contains a further ingredient, optionally in addition to the cosolvent, such further ingredient is selected from a colorant, a flavoring agent, a filler, a chelating agent; or from a colorant, a flavoring agent, and a chelating agent; or from a colorant and a flavoring agent.

### The Alginate

The alginate used according to the present invention is an alginate salt of monovalent cation or a mixture of alginate salts of monovalent cation (e.g. a cation such as Li⁺, Na⁺, K⁺, NH₄⁺, or any other pharmaceutically acceptable monovalent cation) said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol, and said alginate salt of monovalent cation or mixture of alginate salts of monovalent cation being such that a 10% aqueous solution thereof at a temperature of 20° C. has a viscosity of 100-1000 mPas, as measured at a shear rate of 20 rpm by use of a Brookfield viscometer with a spindle No. 2.

An example of such an alginate is Protanal® LFR 5/60 NF, a sodium alginate which may be purchased e.g. from FMC Bio Polymer.

According to the present invention, the alginate salt of monovalent cation or mixture of alginate salts of monovalent cation as defined herein above is used as a film-forming agent. In some embodiments, the film may also contain one or more further film forming agents. However, it is an advantageous feature of the film of the present invention that no such further film forming agents are required in order for the advantageous features of mucoadhesivity and suitable dissolution profile to be achieved.

Consequently, in some embodiments, the mucoadhesive film described herein contains no further film forming agent. In some embodiments, the film contains at most 20% of any further film forming agent, at most 15%, at most 10%, or at most 5% of any further film forming agent, based on the total weight of the dry film. In some embodiments, if the film contains any further film forming agent, such further agent is not polyvinyl alcohol. In some embodiments, if the film contains any further film forming agent is an alginate, e.g. an alginate salt of monovalent ion.

### The Method of Preparation

A method of preparing a mucoadhesive film containing vardenafil or a pharmaceutically acceptable salt thereof is provided herein, which method comprises: admixing, in an aqueous liquid carrier, (i) vardenafil or a pharmaceutically acceptable salt of vardenafil; (ii) at least one C3-C12 polyol; and a pH regulating agent, to obtain a liquid composition having a pH of at least 4.2; admixing the obtained liquid composition having a pH of at least 4.2 with an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol, and said alginate salt of monovalent cation or mixture of alginate salts of monovalent cation being such that a 10% aqueous solution thereof at a temperature of 20° C. has a viscosity of 100-1000 mPas, as measured at a shear rate of 20 rpm by use of a Brookfield viscometer with a spindle No. 2; to obtain a film-forming liquid composition; distributing the obtained film-forming liquid composition onto a solid surface; and permitting the film-forming liquid composition to dry on said surface.

Thus, the process described herein comprises admixing, in an aqueous liquid carrier, (i) vardenafil or a pharmaceutically acceptable salt of vardenafil; (ii) at least one C3-C12 polyol; and a pH regulating agent, to obtain a liquid composition having a pH of at least 4.2.

It should be realized that components (i) and (ii) may be admixed in any order, e.g. (i) may be admixed with the liquid carrier and thereafter (ii) is added, or (ii) may be admixed with the liquid carrier and thereafter (i) is added. In other embodiments, (i) and (ii) may be mixed together and admixed with the liquid carrier, or each component may be separated admixed with liquid carrier and then mixed together. In some embodiments, component (ii) is comprised of more than one C3-C12 polyol, and then these polyols may be admixed in any order with component (i), in a dry state or after admixing (i) and/or (ii) with a liquid carrier.

The pH regulating agent suitably is added to the liquid composition after all of component (i) has been added. In some embodiments, the pH regulating agent is admixed with the liquid carrier containing component (i) before admixing component (ii). Thus, in some embodiments the method comprises admixing, in an aqueous liquid carrier, (i) vardenafil or a pharmaceutically acceptable salt of vardenafil, adding a pH regulating agent; followed by admixing at least one C3-C12 polyol, to obtain a liquid composition having a pH of at least 4.2.

In some embodiments, the pH of the liquid composition containing components (i) and (ii) is determined, and the necessary amount of pH regulating agent then is added, to achieve a liquid composition having a pH of at least 4.2. Generally, before addition of the pH regulating agent, the pH of the liquid composition containing components (i) and (ii) will be lower than about 4.1, e.g. lower than about 3.9, such as about 3.4 to about 4.0.

In some embodiments, the pH regulating agent is admixed with the liquid carrier containing both components (i) and (ii). Thus, in some embodiments the method comprises admixing, in an aqueous liquid carrier, (i) vardenafil or a pharmaceutically acceptable salt of vardenafil and (ii) at least one C3-C12 polyol, followed by adding a pH regulating agent in an amount sufficient to provide a pH of at least 4.2.

The aqueous liquid carrier used in the method preferably is deionized water (of pharmaceutical quality), optionally in admixture with one or more pharmaceutically acceptable organic solvents.

In some embodiments, components (i) and (ii) are present in the liquid mixture at a concentration of from 1 to 20% by weight of component (i), e.g. from 1 to 10% by weight of component (i), or from 1 to 5% by weight of component (i); and from 1 to 20% by weight of component (ii), e.g. from 1 to 10% by weight of component (ii), or from 2 to 10% by weight of component (ii), based on the weight of the composition before admixing the alginate component.

The molar ratio of component (i) to component (ii) generally ranges from about 1:1 to about 1:50, e.g. from about 1:2 to about 1:40, from about 1:2 to about 1:30, such as from about 1:2 to about 1:20, or about 1:2 to about 1:15. In some embodiments, the molar ratio of (i) to (ii) ranges from about from about 1:3 to about 1:40, from about 1:3 to about 1:30, such as from about 1:3 to about 1:20, or about 1:3 to about 1:15. In some embodiments, the molar ratio ranges from about from about 1:4 to about 1:40, from about 1:4 to about 1:30, such as from about 1:4 to about 1:20, or about 1:4 to about 1:15.

For example, in some embodiments, a liquid composition is prepared containing from about 0.01 to about 1 mol/L of component (i), e.g. from about 0.02 to about 0.5 mol/L of component (i), or from about 0.04 to about 0.2 mol/L of component (i), and an amount of component (ii) such as to provide a molar ratio within any of the above indicated ranges.

The pH regulating agent generally is a pharmaceutically acceptable alkaline reacting compound, or mixture of such compounds, e.g. a strong base, such as NaOH or KOH, in particular NaOH. In some embodiments, the pH regulating agent is admixed with the liquid carrier containing component (i). Thus, in some embodiments the method comprises admixing, in an aqueous liquid carrier, (i) vardenafil or a pharmaceutically acceptable salt of vardenafil followed by adding a pH regulating agent and subsequently admixing at least one C3-C12 polyol, to obtain a liquid composition having a pH of at least 4.2.

Before admixing the liquid composition and the alginate component, it is important that the liquid composition has a pH of at least 4.2. In some embodiments, the liquid composition has a pH of from 4.2 to 13, or from 4.2 to 12, or from 4.2 to 11 before admixing with the alginate component. In some embodiments, the pH is at least 4.3, at least 4.4, at least 4.5, at least 4.6, at least 4.7, at least 4.8, at least 4.9, at least 5.0, at least 6.0, or at least 7.0. In some embodiments, the pH is from 4.2 to 11, from 4.2 to 10, from 4.2 to 9, from 4.2 to 8, from 4.2 to 7, or from 4.2 to 6; e.g. from 4.3 to 11, from 4.3 to 10, from 4.3 to 9, from 4.3 to 8, from 4.3 to 7, or from 4.3 to 6; from 4.4 to 11, from 4.4 to 10, from 4.4 to 9, from 4.4 to 8, from 4.4 to 7, or from 4.4 to 6; from 4.5 to 11, from 4.5 to 10, from 4.5 to 9, from 4.5 to 8, from 4.5 to 7, or from 4.5 to 6; from 4.6 to 11, from 4.6 to 10, from 4.6 to 9, from 4.6 to 8, from 4.6 to 7, or from 4.6 to 6; from 4.7 to 11, from 4.7 to 10, from 4.7 to 9, from 4.7 to 8, from 4.7 to 7, or from 4.7 to 6; from 4.8 to 11, from 4.8 to 10, from 4.8 to 9, from 4.8 to 8, from 4.8 to 7, or from 4.8 to 6; or from 4.9 to 6; from 4.9 to 11, from 4.9 to 10, from 4.9 to 9, from 4.9 to 8, from 4.9 to 7, or from 4.9 to 6.

The pH preferably is measured by use of a conventional pH meter, at room temperature (about 18-25° C.).

Once the liquid composition, containing vardenafil or a pharmaceutically acceptable salt thereof, polyol, and optionally any further ingredient, e.g. ingredient (iii) as mentioned herein above, is at the required minimum pH, the liquid composition and the alginate component may be mixed together. This suitably is achieved by adding the alginate to the liquid composition under constant stirring, e.g. with a paddle mixer, until a homogeneous film-forming liquid composition is obtained.

The alginate component preferably is added to the liquid mixture in an amount of about 20% by weight to about 80% by weight of the dry matter (i.e. the ingredients excluding water), e.g. an amount of about 30 to about 70%, or an amount of about 40 to about 60% by weight, or an amount of about 40 to about 50% by weight.

The film-forming liquid composition may suitably be subjected to a de-aeration treatment, e.g. by use of ultrasonic treatment or vacuum treatment, as well-known to the person of ordinary skill in the art, or by simply leaving the composition, preferably under a cover, such as a plastic film, under a sufficient time, e.g. 2 hours to 24 hours, or 4 hours to 12 hours, e.g. about 6-9 hours.

The method further comprises distributing the obtained (optionally de-aerated) film-forming liquid composition onto a solid surface. This step may be performed by simply pouring the composition onto a smooth and even surface, optionally using a drawdown blade. Once the liquid composition has been distributed onto the surface, it is allowed to dry. Drying may be performed at room temperature or at higher than room temperature, and optionally under reduced pressure. For example, the film may be submitted to a drying treatment for a period of 5-24 hours, or longer, in an oven at a temperature of 30-45° C. The film may be considered dry when dry to the touch and/or when essentially no weight loss occurs on further drying.

Drying preferably is effected until the formulation reaches a level of dryness equal to that which it would have in equilibrium with a surrounding atmosphere having a relative humidity of 10 to 40% at 25° C., e.g. 20 to 30% at 25° C., e.g. a water content of about 8% by weight.

To prepare a dry film, the process for preparing a dry film as generally described in WO 2007/073346 may be followed.

For example, the film forming composition may be distributed onto a solid, flat surface as a wet film having a thickness of from e.g. 0.1 to 4 mm, such as 0.2 to 2 mm, e.g. 0.5 to 1.5 mm. The wet film then is allowed to dry on the surface, e.g. at room temperature or in a ventilated oven or drying cabinet at a temperature of 30-60° C., e.g. at a temperature of from 35 to 50° C., e.g. 35 to 45° C., such as about 40° C., for a time period of e.g. 20 to 120 minutes, or from 30 to 60 minutes.

Once dried, the film suitably has a thickness of from about 0.05 mm to about 2 mm, e.g. from about 0.1 mm to about 1.5 mm, or from about 0.2 mm to about 1.4 mm, from about 0.3 mm to about 1.3 mm, from about 0.4 mm to about 1.2 mm, e.g. about 0.5 mm to about 1.1 mm, or about 0.8 mm to about 1 mm. More preferably, the dry film suitably has a thickness from about 0.05 mm to about 1 mm, e.g. from about 0.05 mm to about 0.5 mm, or from about 0.05 mm to about 0.4 mm, from about 0.05 mm to about 0.3 mm, or from about 0.05 mm to about 0.2 mm, or from about 0.1 mm to about 1 mm, e.g. from about 0.1 mm to about 0.5 mm, or from about 0.1 mm to about 0.4 mm, from about 0.1 mm to about 0.3 mm, or from about 0.1 mm to about 0.2 mm.

The dry film is divided into unit pieces of suitable surface area, e.g. 1 cm² to 8 cm², or 1 cm² to 6 cm², or 1 cm² to 4 cm², or 1 cm² to 3 cm². In some embodiments, the surface area of each piece is 1.5 cm² to 6 cm², or 1.5 cm² to 5 cm², or 1.5 cm² to 4 cm², or 1.5 cm² to 3 cm². In some embodiments, the surface area of each piece is 2 cm² to 6 cm², or 2 cm² to 5 cm², or 2 cm² to 4 cm², or 2 cm² to 3 cm².

The film obtained at the end of the drying is a mucoadhesive film comprising

(i) vardenafil or a pharmaceutically acceptable salt of vardenafil;

(ii) at least one C3-C12 polyol; and

an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

In some preferred embodiments, the method of preparing a mucoadhesive film containing vardenafil or a pharmaceutically acceptable salt thereof comprises: admixing, in an aqueous liquid carrier, (i) vardenafil or a pharmaceutically acceptable salt of vardenafil; (ii) at least one C3-C12 polyol; (iii) at least one of DMSO and NMP; and a pH regulating agent, to obtain a liquid composition having a pH of at least 4.2; admixing the obtained liquid composition having a pH of at least 4.2 with an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol, and said alginate salt of monovalent cation or mixture of alginate salts of monovalent cation being such that a 10% aqueous solution thereof at a temperature of 20° C. has a viscosity of 100-1000 mPas, as measured at a shear rate of 20 rpm by use of a Brookfield viscometer with a spindle No. 2; to obtain a film-forming liquid composition; distributing the obtained film-forming liquid composition onto a solid surface; and permitting the film-forming liquid composition to dry on said surface.

In such preferred embodiments, the film obtained at the end of the drying is a mucoadhesive film comprising

(i) vardenafil or a pharmaceutically acceptable salt of vardenafil

(ii) at least one C3-C12 polyol,

(iii) at least one of DMSO and NMP, and

an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

It should be realized that components (i), (ii), (iii) and carrier may be admixed in any order, e.g. (i), (ii), and (iii) may be mixed together and the mixture then admixed with the liquid carrier.

The pH regulating agent suitably is added to the liquid composition after all of component (i) has been added. In some embodiments, the pH regulating agent is admixed with the liquid carrier containing component (i) before admixing components (ii) and (iii). Thus, in some embodiments the method comprises admixing, in an aqueous liquid carrier, (i) vardenafil or a pharmaceutically acceptable salt of vardenafil, adding a pH regulating agent; followed by admixing at least one C3-C12 polyol, and at least one of DMSO and NMP, to obtain a liquid composition having a pH of at least 4.2.

In some embodiments, the pH of the liquid composition containing components (i), (ii) and (iii) is determined, and the necessary amount of pH regulating agent then is added, so as to to achieve a liquid composition having a pH of at least 4.2. Generally, before addition of the pH regulating agent, the pH of the liquid composition containing components (i), (ii) and (iii) will be lower than about 4.1, e.g. lower than about 3.9, such as about 3.4 to about 4.0.

In some embodiments, the pH regulating agent is admixed with the liquid carrier containing both components (i), (ii) and (iii). Thus, in some embodiments the method comprises admixing, in an aqueous liquid carrier, (i) vardenafil or a pharmaceutically acceptable salt of vardenafil; (ii) at least one C3-C12 polyol; and (iii) at least one of DMSO and NMP; followed by adding a pH regulating agent in an amount sufficient to provide a pH of at least 4.2.

The aqueous liquid carrier used in the method preferably is deionized water (of pharmaceutical quality), optionally in admixture with one or more pharmaceutically acceptable organic solvents.

In some embodiments, components (i), (ii) and (iii) are present in the liquid mixture at a concentration of from 1 to 20% by weight of component (i), e.g. from 1 to 10% by weight of component (i), or from 1 to 5% by weight of component (i); from 1 to 20% by weight of component (ii), e.g. from 1 to 10% by weight of component (ii), or from 2 to 10% by weight of component (ii); and from 1 to 15% by weight of component (iii), e.g. from 1 to 10% by weight of component (iii), or from 1 to 8% by weight of component (iii), based on the weight of the composition before admixing the alginate component.

In some embodiments, the components (i) and (iii) are present in weight ratios of (i) to (iii) of from about 10:1 to about 1:1; from about 8:1 to about 1:1, from about 5:1 to about 1:1; or from about 4:1 to about 1:1, or from about 3:1 to about 1:1, e.g. from about 2:1 to about 1:1. In some embodiments, the components (i) and (iii) are present in weight ratios of (i) to (iii) of from about 10:1 to about 2:1; from about 8:1 to about 2:1, from about 5:1 to about 2:1; or from about 4:1 to about 2:1. In some embodiments, the components (i) and (iii) are present in weight ratios of (i) to (iii) of from about 10:1 to about 3:1; from about 8:1 to about 3:1, from about 6:1 to about 3:1; or from about 5:1 to about 3:1. In some embodiments, the components (i) and (iii) are present in weight ratios of (i) to (iii) of from about 10:1 to about 4:1; from about 8:1 to about 4:1, from about 6:1 to about 4:1; or from about 5:1 to about 4:1. In some embodiments, the components (i) and (iii) are present in weight ratios of (i) to (iii) of from about 10:1 to about 5:1; from about 8:1 to about 5:1, from about 7:1 to about 5:1; or from about 6:1 to about 5:1.

In some embodiments, the film obtained at the end of drying is a mucoadhesive film comprising

(i) vardenafil or a pharmaceutically acceptable salt of vardenafil;

(ii) xylitol and optionally at least one further C3-C12 polyol;

(iii) at least one of DMSO and NMP, preferably DMSO; and

an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

In some embodiments, the mucoadhesive film of the invention comprises

(i) vardenafil or a pharmaceutically acceptable salt of vardenafil; e.g vardenafil hydrochloride;

(ii) xylitol, and optionally also sorbitol and glycerol;

(iii) DMSO; and

an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

The film may suitably be provided as unit doses as described herein, e.g. by cutting or punching the dry or semi-dry film.

A film dose unit (or dosage unit) normally contains vardenafil or a pharmaceutically acceptable salt thereof in an amount (expressed as vardenafil) that suitably ranges from 0.5 to 20 mg, from 1 mg to 20 mg, e.g. 2 mg to 20 mg, or from 5 mg to 20 mg. In some embodiments, a film dose unit contains from 0.5 to 10 mg, from 1 mg to 10 mg, e.g. from 1.5 mg to 10 mg, or from 2 mg to 10 mg. In some embodiments the doe unit of active ingredient (expressed as vardenafil) suitably is from 0.5 mg to 5 mg, from 1 mg to 5 mg, from 1.5 mg to 5 mg, or from 2 mg to 5 mg, e.g. 0.5 mg to 4 mg, from 1 mg to 4 mg, from 1.5 mg to 4 mg, or from 2 mg to 4 mg, or from 0.5 mg to 3 mg, from 1 mg to 3 mg, from 1.5 mg to 3 mg, or from 2 mg to 3 mg.

For example, a dose unit may be in the form of a film having a surface area of 1-4 cm² and a thickness of 0.1-1.5 mm, and contain from 1 to 10 mg of vardenafil; e.g. a film having a surface area of 1.5-3 cm² and a thickness of 0.1-1.2 mm, and containing from 1 to 5 mg of vardenafil, or a film having a surface area of 1.5-2 cm² and a thickness of 0.1-1.0 mm, and containing from 1.5 to 3 mg of vardenafil, such as a film having a surface area of about 1.5 cm² and a thickness of about 1.0 mm and containing 2 mg of vardenafil.

In some embodiments, a dose unit is in the form of a film having a surface area of 1-3 cm² and a thickness of 0.1-1.0 mm, containing from 1 to 20 mg of vardenafil.

In some embodiments, a dose unit is in the form of a film having a surface area of 1-3 cm² and a thickness of 0.1-1.0 mm, containing from 1 to 10 mg of vardenafil.

In some embodiments, a dose unit is in the form of a film having a surface area of 1-3 cm² and a thickness of 0.1-1.0 mm, containing from 1 to 5 mg of vardenafil.

In some embodiments, a dose unit is in the form of a film having a surface area of 2-4 cm² and a thickness of 0.1-1.0 mm, containing from 1 to 20 mg of vardenafil.

In some embodiments, a dose unit is in the form of a film having a surface area of 2-4 cm² and a thickness of 0.1-1.0 mm, containing from 1 to 10 mg of vardenafil.

In some embodiments, a dose unit is in the form of a film having a surface area of 2-4 cm² and a thickness of 0.1-1.0 mm, containing from 1 to 5 mg of vardenafil.

In some embodiments, a dose unit is in the form of a film having a surface area of 2-3 cm² and a thickness of 0.1-1.0 mm, containing from 1 to 20 mg of vardenafil.

In some embodiments, a dose unit is in the form of a film having a surface area of 2-3 cm² and a thickness of 0.1-1.0 mm, containing from 1 to 10 mg of vardenafil.

In some embodiments, a dose unit is in the form of a film having a surface area of 2-3 cm² and a thickness of 0.1-1.0 mm, containing from 1 to 5 mg of vardenafil.

In some embodiments, a dose unit is in the form of a film having a surface area of 2-4 cm² and a thickness of 0.1-0.5 mm, containing from 1 to 10 mg of vardenafil, e.g. from 5 to 10 mg of vardenafil.

In some embodiments, a dose unit is in the form of a film having a surface area of 2-4 cm² and a thickness of 0.1-0.3 mm, containing from 1 to 10 mg of vardenafil, e.g. from 5 to 10 mg of vardenafil.

In some embodiments, a dose unit is in the form of a film having a surface area of 2-4 cm² and a thickness of 0.1-0.3 mm, containing from 4 to 8 mg of vardenafil.

In some embodiments, a dose unit is in the form of a film having a surface area about 3 cm² and a thickness of about 0.2 mm, containing from about 1 to about 10 mg of vardenafil, e.g. about 5 to about 8 mg of vardenafil, or about 5 to about 7 mg of vardenafil.

In some embodiments, a dose unit as mentioned herein above contains an ingredient (iii), in an amount of from about 1 to about 10% by weight of the dose unit, or from about 2 to about 8% by weight, or from about 3 to about 6% by weight, e.g. about 3 to about 5% by weight, based on the total weight of the dry film.

For example, in some embodiments, a dose unit is provided in the form of a film having a surface area of about 2-4 cm², and a thickness of about 0.1-0.3 mm, said dose unit containing vardenafil in an amount of about 5 to about 10 mg and containing about 3 to about 5% by weight of ingredient (iii), preferably DMSO.

In some preferred embodiments, a dose unit as mentioned herein above also contains xylitol, e.g. from 1 to 20 mg of xylitol, or from 5 to 10 mg of xylitol, and optionally also contains one or more further C3-C12 polyols, e.g. one or more further C3-C7 polyols, such as glycerol and/or sorbitol.

For example, in some embodiments, a dose unit is provided in the form of a film having a surface area of about 2-4 cm², and a thickness of about 0.1-0.3 mm, said dose unit containing vardenafil or a pharmaceutically acceptable salt thereof in an amount corresponding to about 5 to about 10 mg of vardenafil, xylitol in an amount of about 5 to about 30 mg, e.g. about 5 to about 15 mg, or about 5 to about 10 mg, and further containing about 3 to about 5% by weight of DMSO.

In some further embodiments, a dose unit is provided in the form of a film having a surface area of about 2-4 cm², and a thickness of about 0.1-0.3 mm, said dose unit containing vardenafil or a pharmaceutically acceptable salt thereof in an amount corresponding to about 5 to about 8 mg of vardenafil, xylitol in an amount of about 5 to about 20 mg, e.g. about 5 to about 10 mg, or about 5 to about 8 mg, and further containing about 3 to about 5% by weight of DMSO.

In some embodiments, a dose unit as described herein above, in addition to xylitol also contains one or more further C3-C12 polyols, as defined herein, e.g. selected from the polyols as mentioned herein above. For example, in some embodiments, the dose unit also contains at least one of glycerol and sorbitol, e.g. in a total amount of such further polyol of from about 5 to about 30 mg.

In some embodiments, a dry film of the invention contains from 5 to 40% by weight of (i) vardenafil or a pharmaceutically acceptable salt thereof; from 5 to 40% of (ii) C3-C12 polyol(s); from 0 to 10% of (iii) DMSO and/or NMP; and from 20 to 80% of an alginate salt of monovalent cation or a mixture of alginate salts of monovalent cation, based on the total weight of the dry film.

The amount of vardenafil or salt of vardenafil is expressed herein by weight of the free base vardenafil, independently of whether vardenafil is present as salt or free base in the film.

In some embodiments, a dry film of the invention contains from 10 to 30% by weight of (i) vardenafil or a pharmaceutically acceptable salt thereof; from 10 to 40% of (ii) C3-C12 polyol(s); from 0 to 5% of (iii) DMSO and/or NMP; and from 30 to 70% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 0 to 5% of (iii) DMSO and/or NMP; and from 30 to 60% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 1 to 5% of (iii) DMSO and/or NMP; and from 30 to 60% of alginate, based on the total weight of the dry film.

In some embodiments, a dry film of the invention contains from 20 to 30% by weight of (i) vardenafil or a pharmaceutically acceptable salt thereof; from 20 to 40% of (ii) C3-C12 polyol(s); from 0 to 5% of (iii) DMSO and/or NMP; and from 30 to 50% of alginate, based on the total weight of the dry film.

In some embodiments, a dry film of the invention contains from 5 to 40% by weight of (i) vardenafil or a pharmaceutically acceptable salt thereof; from 5 to 40% of (ii) C3-C12 polyol(s); from 1 to 10% of (iii) DMSO and/or NMP; and from 20 to 80% of alginate, based on the total weight of the dry film.

In some embodiments, a dry film of the invention contains from 10 to 30% by weight of (i) vardenafil or a pharmaceutically acceptable salt thereof; from 10 to 40% of (ii) C3-C12 polyol(s); from 1 to 5% of (iii) DMSO and/or NMP; and from 30 to 70% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 1 to 5% of (iii) DMSO and/or NMP; and from 30 to 60% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 1 to 5% of (iii) DMSO and/or NMP; and from 30 to 60% of alginate, based on the total weight of the dry film.

In some embodiments, a dry film of the invention contains from 20 to 30% by weight of (i) vardenafil or a pharmaceutically acceptable salt thereof; from 20 to 40% of (ii) C3-C12 polyol(s); from 1 to 5% of (iii) DMSO and/or NMP; and from 30 to 50% of alginate, based on the total weight of the dry film.

In some embodiments, a dry film of the invention contains from 5 to 40% by weight of (i) vardenafil or a pharmaceutically acceptable salt thereof; from 5 to 40% of (ii) C3-C12 polyol(s); from 2 to 10% of (iii) DMSO and/or NMP; and from 20 to 80% of alginate, based on the total weight of the dry film.

In some embodiments, a dry film of the invention contains from 10 to 30% by weight of (i) vardenafil or a pharmaceutically acceptable salt thereof; from 10 to 40% of (ii) C3-C12 polyol(s); from 2 to 5% of (iii) DMSO and/or NMP; and from 30 to 70% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 2 to 5% of (iii) DMSO and/or NMP; and from 30 to 60% of alginate, based on the total weight of the dry film.

In some embodiments, a dry film of the invention contains from 15 to 30% by weight of (i) vardenafil or a pharmaceutically acceptable salt thereof; from 20 to 40% of (ii) C3-C12 polyol(s); from 2 to 5% of (iii) DMSO and/or NMP; and from 30 to 60% of alginate, based on the total weight of the dry film.

In the above embodiments, the component (iii) preferably is DMSO. In some embodiments, however, component (iii) is NMP. In some further embodiments, component (iii) is mixture of DMSO and NMP.

In an advantageous embodiment of the invention, the film comprises xylitol and preferably also a component (iii), which preferably is DMSO. It is considered that the component (iii) improves the dissolution properties and bioavailability of vardenafil, while xylitol acts to mask the taste of vardenafil, in addition to being a plasticizer.

The dry dosage units may be packaged into suitable containers, e.g. resealable containers of a water and air tight material suitable for use in packaging of products for human ingestion, e.g. a metallised polyethylene film (Alu/PET). In some embodiments, therefore, a film as described herein is provided in the form of a dosage unit in a wrapper of a preferably air tight material.

The vardenafil formulation of the invention is a water-soluble film, such as a mucoadhesive film, which on application to oral mucosa adheres thereto and dissolves, allowing the vardenafil contained in the film to penetrate the mucosal membrane and enter the blood stream. As noted herein, mucoadhesive films are generally described in PCT/SE2006/050626 (WO 2007/073346).

As used herein, the term mucoadhesive refers generally to the capacity of adhering to a mucous membrane. Thus, when applied to the inside of the mouth, the film of the invention adheres firmly and rapidly and dissolves over a time period of less than a few minutes, e.g. from 1 minute to 10 minutes, or from 2 minutes to 8 minutes, e.g. from 3 minutes to 6 minutes.

The film described herein allows for buccal administration of vardenafil by application of the film described herein to the mucosal surface inside the mouth of an adult patient, preferably a male adult. In some embodiments, therefore, a film as described herein is provided for use in the treatment of sexual dysfunction, e.g. male erectile disorder, by applying the film to the oral mucosal surface of said male and allowing the film to dissolve in contact with the oral mucosal surface. Therefore, also provided herein is a method for the treatment of male sexual dysfunction by transmucosal administration of vardenafil using the film described herein.

Also provided herein is the mucoadhesive film as defined herein above, for use in therapy, e.g. for use in the treatment of sexual dysfunction, preferably male sexual dysfunction, such as erectile disorder of an adult human male. Such use comprises transmucosal, preferably buccal, administration of a dose unit as defined herein above. In some embodiments, the administration may be performed at most once daily. In some embodiments, administration is performed from 5 minutes to 2 hours before sexual activity, e.g. sexual intercourse, e.g. from 10 minutes to 1 hour before sexual intercourse, or from 0.25 hour to 0.5 hour before sexual intercourse.

Also provided herein is the use of the mucoadhesive film as defined herein in the manufacturing of a medicament for the treatment of sexual dysfunction, preferably male sexual dysfunction, such as male erectile disorder. The manufacturing may comprise e.g. cutting or punching suitably sized pieces of the film, marking the dose units with suitable information, e.g. the trade name or the dose strength, packaging the film in suitable containers and/or wrappers, etc.

Also provided herein is the use of the film forming composition comprising components (i), (ii), and optional component (iii), pH regulating agent and alginate salt of monovalent cation or mixture of alginate salts of monovalent cation as defined herein, in the manufacturing of a medicament for the treatment of sexual dysfunction, preferably male sexual dysfunction, such as male erectile disorder. The manufacturing comprises forming a film by a method as described herein.

The film and its preparation and use will be illustrated in the following non-limiting examples.

### EXAMPLE 1

The ingredients listed in Table 1 were used in the indicated amounts.

TABLE 1

| Ingredient | Amount | |
|---|---|---|
| Vardenafil hydrochloride (“VDL-HCL”) | 11 | g |
| Xylitol | 15 | g |
| Glycerol | 10 | g |
| Sorbitol | 10 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M), 2-3 ml | to pH 5 |

VDL-HCl, glycerol, sorbitol and Na₂-EDTA were mixed together and the mixture was dissolved in water. Xylitol was added. The liquid solution was adjusted to pH 5 by addition of NaOH (pH was measured at room temperature using a laboratory pH meter). Alginate was admixed and stirring was performed until a homogeneous liquid mixture was obtained. The liquid mixture was de-aerated for about 12 hours, by simply leaving the mixture in a beaker covered by a plastic film. The de-aerated liquid mixture was distributed onto a solid surface, and allowed to dry in an oven at 40° C. for 40 minutes. A dry film was obtained having a thickness of about 0.2 mm. The film had a smooth and even surface. The film was cut into pieces of 1.5 cm², each piece containing 2 mg of vardenafil hydrochloride.

When applied to the inside of the mouth (buccal application), the film adhered more or less instantly and dissolved over a time period of 3-6 minutes, to provide transmucosal (buccal) administration of vardenafil.

### EXAMPLE 2

Using the ingredients and amounts listed in the above Table 1, a film was prepared as follows: VDL-HCL was dissolved in water. To the aqueous VDL-HCL solution, glycerol, sorbitol, Na₂-EDTA, and xylitol were added in the indicated order, with continued stirring homogeneity in between the addition of each separate ingredient. The obtained solution was adjusted to pH 5 by addition of NaOH and then alginate was added. The liquid mixture was de-aerated for about 12 hours. The de-aerated liquid mixture was distributed onto a solid surface, and allowed to dry in an oven at 40° C. for 40 minutes. A dry film was obtained having a thickness of about 1 mm. The film had a smooth and even surface.

### EXAMPLE 3

Using the ingredients and amounts listed in the above Table 1, a film was prepared as follows: VDL-HCL and xylitol were dissolved in water. Glycerol, sorbitol, and Na₂-EDTA were added to the solution. The obtained solution was adjusted to pH 5 by addition of NaOH and then alginate was added. The liquid mixture was de-aerated for 12 hours. The de-aerated mixture was distributed onto a solid surface, and the wet film was allowed to dry in an oven at 40° C. for 40 minutes. A dry film was obtained having a thickness of about 0.2 mm. The film had a smooth and even surface.

### EXAMPLE 4

The ingredients listed in Table 2 were used in the indicated amounts.

TABLE 2

| Ingredient | Amount | |
|---|---|---|
| Vardenafil hydrochloride (“VDL-HCL”) | 11 | g |
| Glycerol | 10 | g |
| Sorbitol | 10 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M), 2-3 ml | to pH 5 |

VDL-HCl, glycerol, sorbitol and Na₂-EDTA were mixed and the mixture was dissolved in water. The aqueous solution was adjusted to pH 5 by addition of NaOH. Alginate was admixed and stirring was performed until a homogeneous liquid mixture was obtained. The liquid mixture was de-aerated for about 12 hours. The de-aerated liquid mixture was distributed onto a solid surface, and allowed to dry in an oven at 40° C. for 40 minutes. A dry film was obtained having a thickness of about 0.2 mm. The film had a smooth and even surface.

### EXAMPLE 5

The ingredients listed in Table 3 were used in the indicated amounts.

TABLE 3

| Ingredient | Amount | |
|---|---|---|
| Vardenafil hydrochloride (“VDL-HCL”) | 11 | g |
| Glycerol | 15 | g |
| Sorbitol | 15 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M), 2-3 ml | to pH 5 |

VDL-HCl, glycerol, sorbitol and Na₂-EDTA were mixed and the mixture was dissolved in water. The aqueous solution was adjusted to pH 5 by addition of NaOH. Alginate was admixed and stirring was performed until a homogeneous liquid mixture was obtained. The liquid mixture was de-aerated for about 12 hours. The de-aerated liquid mixture was distributed onto a solid surface, and allowed to dry in an oven at 40° C. for 40 minutes. A dry film was obtained having a thickness of about 0.2 mm. The film had a smooth and even surface.

### EXAMPLE 6

The ingredients listed in Table 4 were used in the indicated amounts.

TABLE 4

| Ingredient | Amount | |
|---|---|---|
| Vardenafil hydrochloride (“VDL-HCL”) | 11 | g |
| Xylitol | 15 | g |
| Protanal ® LF 5/60 sodium alginate | 50 | g |
| Deionized water (room temperature) | 300 | ml |

| | |
|---|---|
| NaOH (4M), 2-3 ml | to pH 5 |

VDL-HCl and xylitol were mixed and the mixture was dissolved in water. The aqueous solution was adjusted to pH 5 by addition of NaOH. Alginate was admixed and stirring was performed until a homogeneous liquid mixture was obtained. The mixture was de-aerated for about 12 hours. The de-aerated mixture was distributed onto a solid surface, and allowed to dry in an oven at 40° C. for 40 minutes. A dry film was obtained having a thickness of about 0.2 mm. The film had a smooth and even surface.

### EXAMPLE 7

The ingredients listed in Table 5 were used in the indicated amounts.

TABLE 5

| Ingredient | Amount | |
|---|---|---|
| Vardenafil hydrochloride (“VDL-HCL”) | 11 | g |
| Xylitol | 15 | g |
| Protanal ® LF 5/60 sodium alginate | 50 | g |
| Deionized water (room temperature) | 300 | ml |

| | |
|---|---|
| NaOH (4M), 2-3 ml | to pH 5 |

VDL-HCl was dissolved in water. To the aqueous VDL-HCl solution, xylitol was added. The obtained solution was adjusted to pH 5 by addition of NaOH. Alginate was admixed and stirring was performed until a homogeneous liquid mixture was obtained. The mixture was de-aerated for about 12 hours. The de-aerated mixture was distributed onto a solid surface, and allowed to dry in an oven at 40° C. for 40 minutes. A dry film was obtained having a thickness of about 0.2 mm. The film had a smooth and even surface.

### EXAMPLE 8

Using the ingredients and amounts listed in the above Table 5 a film was prepared as follows: Xylitol was dissolved in water. To the aqueous xylitol solution, VDL-HCL was added. The obtained solution was adjusted to pH 5 by addition of NaOH. Alginate was admixed and stirring was performed until a homogeneous liquid mixture was obtained. The mixture was de-aerated for about 12 hours. The de-aerated mixture was distributed onto a solid surface, and allowed to dry in an oven at 40° C. for 40 minutes. A dry film was obtained having a thickness of about 0.2 mm. The film had a smooth and even surface.

### EXAMPLE 9

The ingredients listed in Table 6 were used in the indicated amounts.

TABLE 6

| Ingredient | Amount | |
|---|---|---|
| Vardenafil hydrochloride (“VDL-HCL”) | 11 | g |
| Xylitol | 15 | g |
| Glycerol | 10 | g |
| Sorbitol | 10 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M) | to pH 4.25 |

VDL-HCL, xylitol, glycerol, sorbitol, and Na₂-EDTA were mixed and dissolved in water. The pH of the solution was adjusted to pH=4.25 by addition of 2.8 mL of 4M NaOH. To this solution the alginate salt was added and the mixture was stirred for approx. 60 min. The liquid mixture was de-aerated for about 12 hours. The mixture was distributed onto a solid surface, and allowed to dry in an oven at 40° C. for 40 minutes. A dry film was obtained having a thickness of about 0.2 mm. The film had a smooth and even surface.

### EXAMPLE 10

Example 9 was repeated with the difference that the pH was adjusted to pH 4.5. A dry film was obtained having a thickness of about 0.2 mm. The film had a smooth and even surface.

### EXAMPLE 11

Example 9 was repeated with the difference that the pH was adjusted to pH 5. A dry film was obtained having a thickness of about 0.2 mm. The film had a smooth and even surface.

### EXAMPLE 12

Example 9 was repeated with the difference that the pH was adjusted to pH 7. A dry film was obtained having a thickness of about 0.2 mm. The film had a smooth and even surface.

### EXAMPLE 13

The ingredients listed in Table 7 were used in the indicated amounts.

TABLE 7

| Ingredient | Amount | |
|---|---|---|
| Vardenafil hydrochloride (“VDL-HCL”) | 11 | g |
| Xylitol | 15 | g |
| Glycerol | 10 | g |
| Sorbitol | 10 | g |
| DMSO | 3 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M) | to pH 7 |

VDL-HCl, xylitol, glycerol, sorbitol, DMSO and Na₂-EDTA were mixed together and the mixture was dissolved in water and mixed for at least 30 minutes using a paddle mixer. The liquid solution was adjusted to pH 7 by addition of NaOH (pH was measured at room temperature using a laboratory pH meter). Alginate was admixed and stirring was performed until a homogeneous liquid mixture was obtained. The liquid mixture was de-aerated for about 12 hours, by simply leaving the mixture in a beaker covered by a plastic film. The de-aerated liquid mixture was distributed onto a solid surface, using a ZUA applicator (slit height 1 mm) and allowed to dry in an oven at 40° C. for 40 minutes. A dry film was obtained having a thickness of about 0.2 mm. The film had a smooth and even surface.

The film was cut into pieces of 1.5×2 cm², each piece containing approximately 5 mg of vardenafil hydrochloride. The amount of DMSO in the film was about 3.1% by weight. Each film piece was packaged into an AluPet bag and the AluPet bag was heat sealed.

### EXAMPLES 14-20

The procedure of Example 13 was followed, using the same ingredients, but for the cosolvent (component (iii), the type and amount of which was varied. The films of Examples 14-20 were obtained, as shown in Table 8.

TABLE 8

| | | Added amount of | Amount of cosolvent |
|---|---|---|---|
| | | cosolvent | in dry film |
| EXAMPLE | Cosolvent | (g) | (% by weight) |
| 14 | DMSO | 4 | 3.9 |
| 15 | DMSO | 6 | 5.7 |
| 16 | DMSO | 8 | 7.8 |
| 17 | NMP | 3 | 3.1 |
| 18 | NMP | 4 | 3.9 |
| 19 | NMP | 6 | 5.7 |
| 20 | NMP | 8 | 7.8 |

The films of Examples 14 to 20 had smooth and even surfaces and a thickness of about 0.2 mm.

The vardenafil content (unit dose) in the samples (3 cm²) of the films prepared in Examples 13, 16, 17 and 20, was determined by UV-VIS spectrometry at 250 nm. The results are shown in Table 9.

TABLE 9

| EXAMPLE | Unit dose of vardenafil (mg) |
|---|---|
| 13 | 4.5 |
| 16 | 4.5 |
| 17 | 4.6 |
| 20 | 4.7 |

### EXAMPLE 21

Example 17 was repeated, but the pH was adjusted to pH 4.4. A dry film was obtained having a thickness of about 0.2 mm. The film had a smooth and even surface.

### EXAMPLE 22

Example 13 was repeated but DMSO (3 g) and NMP (3 g) were added. The amounts of DMSO and NMP in the dry film were 3.1% each. A dry film was obtained having a thickness of about 0.2 mm. The film had a smooth and even surface.

### Comparative Example 1 (Not According to the Invention)

The ingredients listed in Table 10 were used in the indicated amounts.

TABLE 10

| Ingredient | Amount | |
|---|---|---|
| Vardenafil hydrochloride (“VDL-HCL”) | 11 | g |
| Xylitol | 15 | g |
| Protanal ® LF 5/60 sodium alginate | 50 | g |
| Deionized water (room temperature) | 300 | ml |

Xylitol was dissolved in water. To the aqueous xylitol solution, VDL-HCl was added. The obtained liquid composition had a pH of 3.5. To this solution, alginate was added. The viscosity of the liquid mixture increased substantially and became jelly like with granules visible to the naked eye. The mixture was distributed onto a solid surface, and allowed to dry in an oven at 40° C. for 40 minutes. The obtained dry film was disrupted by irregular holes all over the surface.

### Comparative Example 2 (Not According to the Invention)

Using the ingredients and amounts listed in the above Table 9 a film was prepared as follows: VDL-HCl was dissolved in water. To the aqueous VDL-HCl solution, xylitol was added. The obtained solution had a pH of 3.5. Alginate was added to the solution. The viscosity of the liquid mixture increased substantially and became jelly like with granules visible to the naked eye. The mixture was distributed onto a solid surface, and allowed to dry in an oven at 40° C. for 40 minutes. The obtained dry film was disrupted by irregular holes all over the surface.

### Comparative Example 3 (Not According to the Invention)

Using the ingredients and amounts listed in the above Table 9 a film was prepared as follows: VDL-HCl and xylitol were mixed together and the mixture was dissolved in water, to give a solution having pH 3.5. To the obtained solution, alginate was added. The viscosity of the liquid mixture increased substantially and became jelly like with granules visible to the naked eye. The mixture was distributed onto a solid surface, and allowed to dry in an oven at 40° C. for 40 minutes. The obtained dry film was disrupted by irregular holes all over the surface.

Comparative Examples 1 to 3 show that an alginate film of inferior quality is obtained when adding alginate to a liquid solution of vardenafil and a polyol as defined herein, at an acidic pH of 3.5.

### Comparative Example 4 (Not According to the Invention)

The ingredients listed in Table 11 were used in the indicated amounts.

TABLE 11

| Ingredient | Amount | |
|---|---|---|
| Xylitol | 15 | g |
| Glycerol | 10 | g |
| Sorbitol | 10 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| HCl (×1M) | to pH 3.5 |
| NaOH (4M) | to pH 5 |

Xylitol, glycerol, sorbitol and Na₂-EDTA were mixed together and the mixture was dissolved in water. The pH of the liquid solution was adjusted to pH 3.5 by addition of HCl. To the solution, alginate was added. The viscosity of the liquid mixture increased substantially and the mixture became jelly like with granules visible to the naked eye. To the jelly like, granular mixture obtained, NaOH was added until the mixture reached pH 5. The jelly like appearance with visible granules disappeared and the liquid mixture became homogenous to the naked eye. The liquid mixture was de-aerated for about 12 hours. The de-aerated liquid mixture was distributed onto a solid surface, and allowed to dry in an oven at 40° C. for 40 minutes. A dry film was obtained having a thickness of about 0.2 mm. The film had a smooth and even surface.

### Comparative Example 5 (Not According to the Invention)

The ingredients listed in Table 12 were used in the indicated amounts.

TABLE 12

| Ingredient | Amount | |
|---|---|---|
| Vardenafil hydrochloride (“VDL-HCL”) | 11 | g |
| Xylitol | 15 | g |
| Glycerol | 10 | g |
| Sorbitol | 10 | g |
| Na₂-EDTA (0.1M) | 18 | ml |
| Protanal ® LF 5/60 sodium alginate | 50 | g |
| Deionized water (room temperature) | 300 | ml |

| | |
|---|---|
| NaOH (4M) | to pH 5 |

VDL-HCL, xylitol, glycerol, sorbitol, Na₂-EDTA and alginate were mixed and the mixture was dissolved in water. A jelly like mixture containing granules visible to the eye was obtained. The mixture was adjusted to pH 5 by addition of NaOH, but the appearance remained jelly like with granules visible to the naked eye. The liquid mixture was de-aerated for about 12 hours. The mixture was distributed onto a solid surface, and allowed to dry in an oven at 40° C. for 40 minutes. The obtained dry film was disrupted by irregular holes all over the surface.

Comparative Examples 4 and 5 show that in the absence (Comparative Ex. 4) of vardenafil, the gelling of the film forming liquid mixture at a pH of 3.5 may be reversed by raising the pH, whereas the gelling is irreversible in the presence of vardenafil (Comparative Ex. 5).

### Dissolution Tests

The dissolution rates of films without (Examples 11 and 12) or with cosolvent (Examples 13-21) were tested. The dissolution tests were performed according to Eur. Ph. method 2.9.3.

Briefly:
Dissolution apparatus 1, basket method
Films were weighed before positioned vertically in the basket
Rotation speed: 50 rpm
500 mL media containing 0.9% NaCl
Temperature: 37° C.
Sample volume: 5 mL
Filtration of samples: 0.45 μm cellulose acetate filter
Sampling points: 2, 6, 10, 15, 20, 25, 35, 45, 60, 75, 90 and 120 min.

Determination of vardenafil concentrations was done by LC/UV analysis.

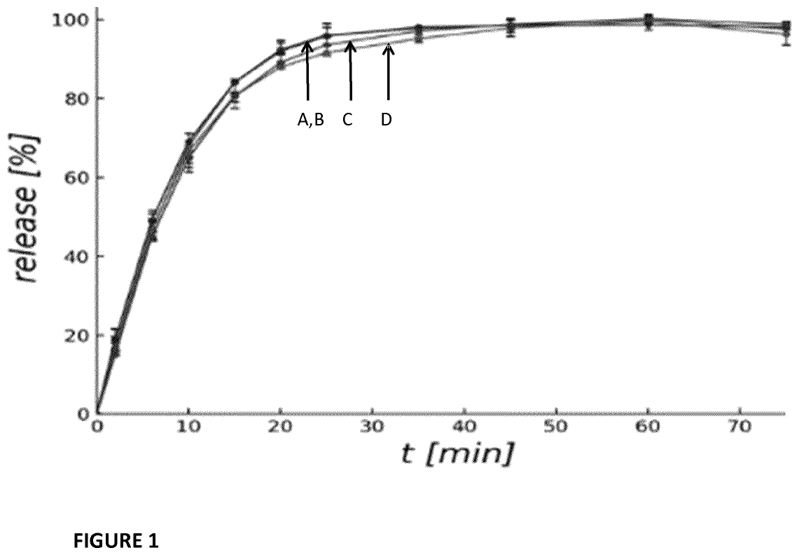

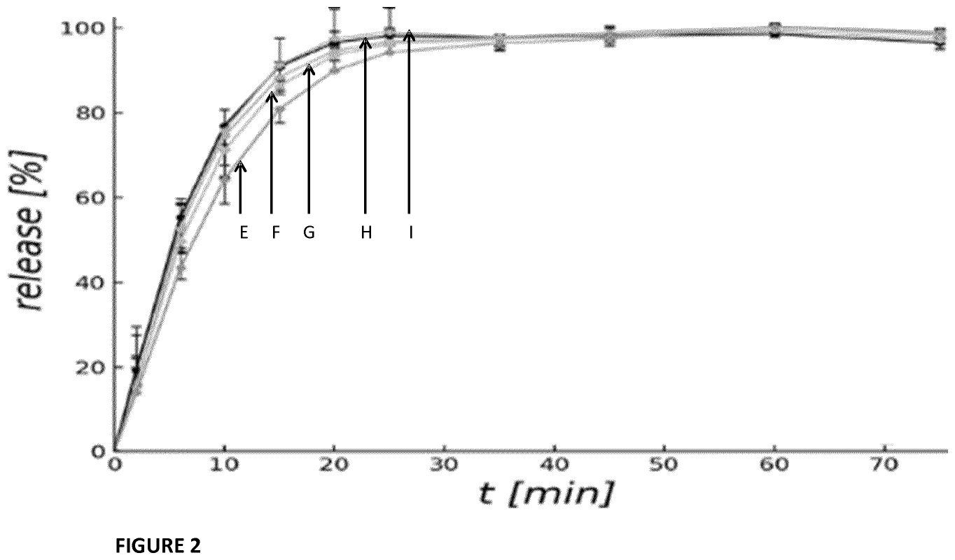

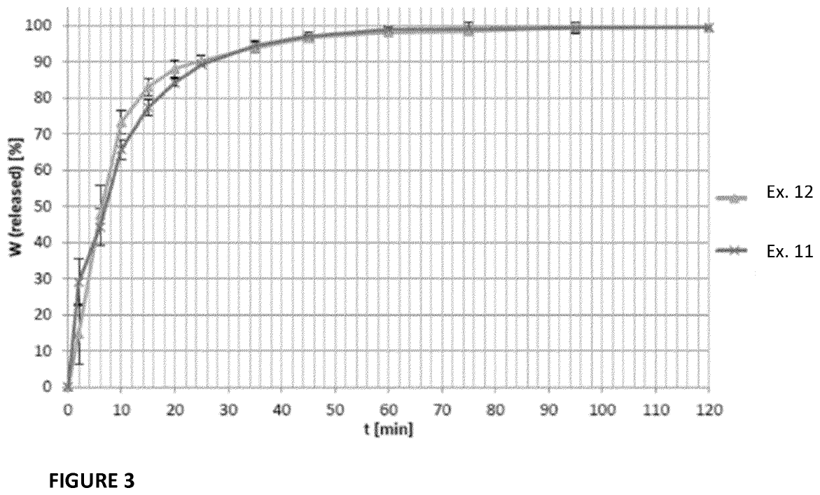

Dissolution rates of formulations containing DMSO or NMP showed very similar curves (FIGS. 1 and 2). The time for complete dissolution of vardenafil is approx. 30 min, which is about half the time required for dissolution without cosolvent (FIG. 3).

### Biological Assays

### Pharmaco-Kinetic Uptake Study in Dogs

Briefly, samples (3 cm²) of some of the Example were administered to the internal mucosa 4 dogs were used and each dog received all each preparation, sequentially, with a washout period of at least 3 days between each administration. Blood was sampled at: pre-dose, 3, 10, 20, 30, 45, 60, min and then 1.5, 2, 4 and 8 hours after administration. Blood samples were collected in K2-EDTA vacuum tubes. Plasma was collected after centrifugation and kept at −20° C., until analyzed.

### Analysis of Dog Plasma Samples

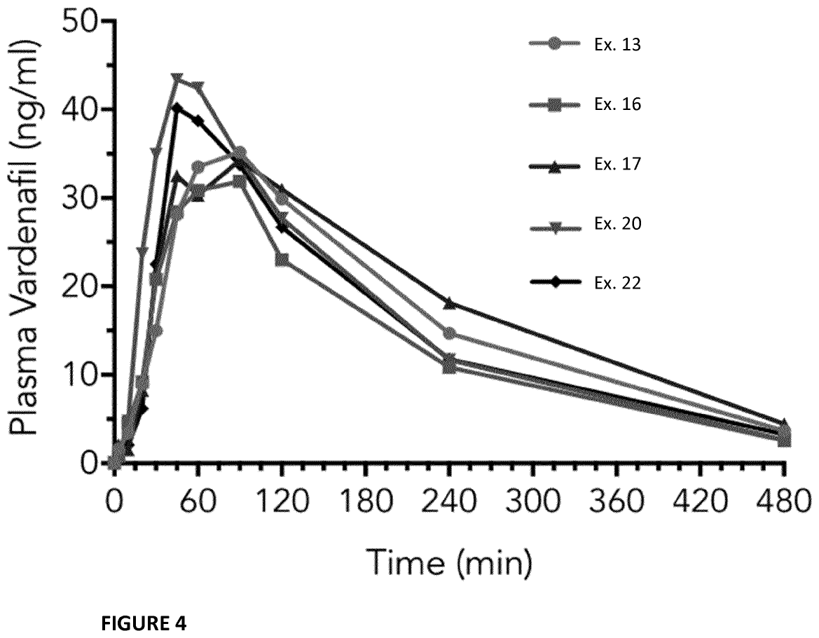

Vardenafil was extracted from dog plasma samples using liquid-liquid-extraction. LC/MS analysis was done on a Waters Acquity system coupled to a Waters XEVO TQS-Micro. Column used was a Waters Acquity UPLC BEH C18 1.7 μm, 2.1×50 mm. The results are shown in FIG. 4 and in Table 13.

TABLE 13

| | | cosolvent | | AUC_{0-8 h} | Cₘₐₓ | tₘₐₓ |
|---|---|---|---|---|---|---|
| EXAM- | | (% by | Dose** | ng/ml*min | ng/ml | min |
| PLE | pH* | weight) | mg | (SD) | (SD) | (SD) |
| 9 | 4.25 | — | 5.1 | 5103 | 25.25 | 71 |
| | | | | (2177) | (7.80) | (33) |
| 10 | 4.5 | — | 5.3 | 5069 | 27.88 | 75 |
| | | | | (1450) | (8.89) | (30) |
| 11 | 5 | — | 5.0 | 5268 | 25.90 | 60 |
| | | | | (1836) | (8.54) | (6) |
| 12 | 7 | — | 5.0 | 6314 | 31.85 | 71 |
| | | | | (978) | (10.57) | (33) |
| 13 | 7 | DMSO (3.1) | 4.5 | 7874 | 37.50 | 71 |
| | | | | (3674) | (16.4) | (23) |
| 16 | 7 | DMSO (7.8) | 4.5 | 7574 | 35.65 | 71 |
| | | | | (2361) | (9.20) | (23) |
| 17 | 7 | NMP (3.1) | 4.6 | 8694 | 42.18 | 75 |
| | | | | (3172) | (10.6) | (37) |
| 20 | 7 | NMP (7.8) | 4.7 | 7856 | 45.08 | 49 |
| | | | | (2104) | (9.98) | (8) |
| 22 | 7 | DMSO (3.1) + | 5.3 | 7361 | 41.28 | 53 |
| | | NMP (3.1) | | (2697) | (15.57) | (9) |

*pH measured in film forming solution before casting
**Dose of vardenafil in 3 cm² film sample.

The PK-profiles obtained from the uptake study in Beagle Dogs showed similar profiles (Cₘₐₓ, AUC_{0-8 h} and tₘₐₓ) for formulations containing DMSO or NMP. No major differences between the two cosolvents were seen. The exception is a shorter tₘₐₓ for preparations at 7.8% NMP, viz. about 20 minutes faster (50 min vs. 70 min). The observed uptake data (Cₘₐₓ and AUC_{0-8 h}) were higher for all cosolvent-containing preparations. Except for the tₘₐₓ for the preparation at 7.8% NMP, no change in tₘₐₓ was seen between preparations with and without cosolvent.

Together, the data obtained show an increase in the transmucosal uptake of vardenafil in the presence of a cosolvent for vardenafil.

1. A method of preparing a mucoadhesive film containing vardenafil or a pharmaceutically acceptable salt of vardenafil, comprising
admixing (i) vardenafil or a pharmaceutically acceptable salt of vardenafil; (ii) at least one C3-C12 polyol; an aqueous liquid carrier; and a pH regulating agent, to obtain a liquid composition having a pH of at least 4.2;
admixing the obtained liquid composition, having a pH of at least 4.2, with an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol, and said alginate salt of monovalent cation or mixture of alginate salts of monovalent cation being such that a 10% aqueous solution thereof at a temperature of 20° C. has a viscosity of 100-1000 mPas, as measured at a shear rate of 20 rpm by use of a Brookfield viscometer with a spindle No. 2; to obtain a film-forming liquid composition,
distributing the obtained film-forming liquid composition onto a solid surface; and permitting the film-forming liquid composition to dry on said surface.

2. The method of claim 1, wherein component (ii) comprises xylitol.

3. The method of claim 1, wherein the pH regulating agent is an alkaline reacting hydroxide of a monovalent metal cation.

4. The method of claim 1, wherein enough pH regulating agent is admixed to obtain a liquid composition having a pH of at least 4.5.

5. The method of claim 1, comprising further admixing a component (iii) selected from dimethylsulfoxide, N-methyl-2-pyrrolidone and a mixture of thereof.

6. The method of claim 5, wherein component (iii) is dimethylsulfoxide.

7. The method of any claim 1, comprising dividing the film into dosage units.

8. A mucoadhesive film comprising
(i) vardenafil or a pharmaceutically acceptable salt of vardenafil,
(ii) at least one C3-C12 polyol, and
an alginate salt of monovalent cation or a mixture of alginate salts of monovalent cation, said alginate salt of monovalent cation or mixture of alginate salts of monovalent cation having a mean guluronate (G) content of from 50 to 85% by weight, a mean mannuronate (M) content of from 15 to 50% by weight, a mean molecular weight of from 30,000 g/mol to 90,000 g/mol and being such that a 10% aqueous solution thereof at a temperature of 20° C. has a viscosity of 100-1000 mPas, as measured at a shear rate of 20 rpm by use of a Brookfield viscometer with a spindle No. 2.

9. The film of claim 8, further comprising a component (iii) selected from dimethylsulfoxide, N-methyl-2-pyrrolidone and a mixture of thereof.

10. The film of claim 8, comprising, by total weight of the film:
(i) vardenafil or a pharmaceutically acceptable salt of vardenafil, in an amount of from 5 to 40% by weight;
(ii) at least one C3-C12 polyol, in an amount of from 5 to 40% by weight;
(iii) dimethylsulfoxide, N-methyl-2-pyrrolidone, or a mixture thereof, in an amount of from 0 to 10% by weight; and
the alginate salt of monovalent cation or mixture of alginate salts of monovalent cation in an amount of from 20 to 80% by weight.

11. The film of claim 10, comprising, by total weight of the film:
(i) vardenafil or a pharmaceutically acceptable salt of vardenafil, in an amount of from 15 to 30% by weight;
(ii) at least one C3-C12 polyol, in an amount of from 20 to 40% by weight;
(iii) dimethylsulfoxide, N-methyl-2-pyrrolidone, or a mixture thereof, in an amount of from 0 to 5% by weight; and
the alginate salt of monovalent cation or mixture of alginate salts of monovalent cation in an amount of from 30 to 60% by weight.

12. The film of claim 10, wherein the component (iii) is present in an amount of at least 1% by weight.

13. The film of claim 12, wherein the component (iii) is present in an amount of at least 3% by weight.

14. The film of claim 9, wherein the component (iii) is dimethylsulfoxide.

15. The film of claim 8, wherein component (ii) comprises xylitol.

16. The film according to claim 8, in the form of a dose unit containing from 1 mg to 20 mg of vardenafil, in the form of a free base or a pharmaceutically acceptable salt.

17. (canceled)

18. (canceled)

19. The method of claim 2, further comprising admixing a component (iii) selected from dimethylsulfoxide, N-methyl-2-pyrrolidone and a mixture of thereof.

20. The film of claim 12, wherein component (ii) comprises xylitol.

21. A method for the treatment of a male sexual dysfunction, comprising administering a film according to claim 8 to a male in need of such treatment.
